SEQUENCE LISTING (2 sequences)

SEQ ID NO: 1 - DNA, 1803 bp, unidentified
  source 1..1803: mol_type = unassigned DNA; note = Artificial sequence
atgaaacacaccattagtaccttaactgccctgctgctatgcagctcgtcgtttgccgccgactgggacggcttacctgtccccgccgatgccggcagcggcaacacctggcaactgcaaagcaacgtctcggacgactttaactactcggccccggccaatggtaaaagcgcagccttttacgatcgctggtccgagggttttattaacgcctggcaaggcccgggcctaaccgactaccacaaccccaactcgcgggttgaaaacggcgagctggtgattcaagccacccgcaaaccgggcaccaatcaggtgtacaccggcgccgtgcacaccaacgacagcatccagtacccggtttacatcgaaaccagcagtaaaattatggatcaggtgctggctaacgccgtgtggatgttaagcagtgattccaccgaagagatcgacattgtcgaggcttacggcagcagtcgccccgatcaaacctggtttgccgagcgtatgcacttagcccaccacgtgtttatccgcgacccgtttcaggattaccaacctaaagacgccggcgcctggtacgccgacggccgcttatggcgtgaccaatacagccgggttggggtttactggcgcgatccttggcacctggagtattacatcgacggccaattggtacgcaccgtatccggtgtagacatgattgacccttacggctacaccaacggcaacggtttaagcaaacctatgcaaattatcgttgatgccgaagatcaggactggcgctcagataacggtattatcgcgactgatgccgatcttgccgacagcagcaaaaaccagttttatgtcgattggattcgcgtttataaaccggtaccagatgccaacggtggcggagataacggcggcgataatggtggcgataacggaggtgacaatggcggcgataacgacattaccagcagcgtcgactttgatagcttttttgcgaccggcaaagacggcagcgccgttgccggcgacagcgttaatggctttaacccctcgggtaatggcaacatcaactacaacaccgtaggcgattgggccgagtacagcatcaatctacccgaggccggtgagtaccgtttagagctggacacggcctcgacagtgagcacaggcctcggcgccgacatcagtatcgacgacgtgtttgtcggcacagttgctatctcgcaaactggcggctgggagagctatcaaacctttagccttgccaacaccatcaacatcggtgccggcacccacacactgcgcgtacaaagcgcaggaagctcgccgtggcagtggaacggcaacgcgatccgcatggttaaagtgggcgaaggctcaagcaacaaccaaacaaccacacctacaccaagcgaaatgatcagcctggaggccgaaagctttaacagcaccggtgggccgtacgacgggtttcaaacttatacccaaagcggtatcacagctaccaactacaaccagcgtggcgactacgccgagtacaccctgtcggtacctaccgcaggcaactacaacgtgagtgctattgtggcgactcccgaaagcggtgccgccatgacgctaacgctaaacggcaacgcgctggttagcttagacgtaccctcaaccggcggctggaacacctttaccgaggttaacgcaagcggcgccgtggcgttgcccgcgggcactcacaccctaagagtaaccagctccggcaacaccgccaacacctgggagtggaacgccgatcgctttatctttacaccgcaataa

SEQ ID NO: 2 - protein, 600 aa, unidentified
  source 1..600: note = Artificial sequence
MKHTISTLTALLLCSSSFAADWDGLPVPADAGSGNTWQLQSNVSDDFNYSAPANGKSAAFYDRWSEGFINAWQGPGLTDYHNPNSRVENGELVIQATRKPGTNQVYTGAVHTNDSIQYPVYIETSSKIMDQVLANAVWMLSSDSTEEIDIVEAYGSSRPDQTWFAERMHLAHHVFIRDPFQDYQPKDAGAWYADGRLWRDQYSRVGVYWRDPWHLEYYIDGQLVRTVSGVDMIDPYGYTNGNGLSKPMQIIVDAEDQDWRSDNGIIATDADLADSSKNQFYVDWIRVYKPVPDANGGGDNGGDNGGDNGGDNGGDNDITSSVDFDSFFATGKDGSAVAGDSVNGFNPSGNGNINYNTVGDWAEYSINLPEAGEYRLELDTASTVSTGLGADISIDDVFVGTVAISQTGGWESYQTFSLANTINIGAGTHTLRVQSAGSSPWQWNGNAIRMVKVGEGSSNNQTTTPTPSEMISLEAESFNSTGGPYDGFQTYTQSGITATNYNQRGDYAEYTLSVPTAGNYNVSAIVATPESGAAMTLTLNGNALVSLDVPSTGGWNTFTEVNASGAVALPAGTHTLRVTSSGNTANTWEWNADRFIFTPQ

(19) United States
(12) Patent Application Publication
(10) Pub. No.: US 2024/0269058 A1
(43) Pub. Date: Aug. 15, 2024

(54) AGAR HYDROLYSIS PRODUCT HAVING HYALURONIDASE INHIBITORY ACTIVITY AND USES THEREOF

(71) Applicant: KOREA INSTITUTE OF OCEAN SCIENCE & TECHNOLOGY, Busan (KR)

(72) Inventors: Chul Hong OH, Jeju-do (KR); Young Deuk LEE, Jeju-do (KR); Eun Young JO, Jeju-do (LR); Tae Yang EOM, Jeju-do (KR); Ye Hui GANG, Jeju-do (KR)

(21) Appl. No.: 18/527,564

(22) Filed: Dec. 4, 2023

(30) Foreign Application Priority Data
Dec. 9, 2022 (KR) 10-2022-0171948

(51) Int. Cl. A61K 8/73; A61Q 19/08; C12N 9/24; C12P 19/04

(52) U.S. Cl. CPC A61K 8/73; A61Q 19/08; C12N 9/2402; C12P 19/04; C12Y 302/01

(57) ABSTRACT

The present disclosure relates to an agar hydrolysis product having hyaluronidase inhibitory activity and uses thereof. According to the present disclosure, the agar partial hydrolysis product excellently inhibits the activity of hyaluronidase, which degrades hyaluronic acid (HA), an important factor in skin moisturization, and thus may be variously used in cosmetics, food, or medicine to maintain and enhance skin moisturization, maintain skin elasticity, improve skin wrinkles, as well as promote skin regeneration and prevent aging.

### CROSS-REFERENCE TO RELATED APPLICATIONS

This application is based on and claims priority from Korean Patent Application No. 10-2022-0171948, filed on Dec. 9, 2022, with the Korean Intellectual Property Office, the disclosure of which is incorporated herein in its entirety by reference.

### SUBMISSION OF SEQUENCE LISTING

The instant application contains a Sequence Listing which has been submitted electronically in .xml format and is hereby incorporated by reference in its entirety. Said .xml file, created on Nov. 30, 2023 is named OP-23120-US_Seq_Listing.xml, and is 5000 bytes in size. The content of the .xml file incorporated herein by reference is a computer readable form (CRF) of four sequences:
SEQ ID NO.1 is the nucleic acid sequence of rGaa16B.
SEQ ID NO.2 is the amino acid sequence of rGaa16B.

### TECHNICAL FIELD

The present disclosure relates to an agar hydrolysis product having hyaluronidase inhibitory activity and uses thereof.

### BACKGROUND

The skin is divided into three layers of epidermis, dermis, and subcutaneous fat tissue sequentially from the outside, and functions to protect the human body from physical and chemical stimulation from the external environment. In particular, the skin plays an important role in controlling about 65 to 70% of the moisture contained in the human body, that is, such a moisture that helps in delivering various biologically active substances necessary for the human body, and maintaining the skin in a soft and moist state, to be evaporated out of the body. In addition, the epidermis is divided into the stratum corneum, stratum granulosum, stratum spinosum, and stratum basale from the outside, and cells of the stratum corneum act like bricks, and the intercellular lipids between keratinocytes act like mortar to form a skin barrier. The stratum corneum of the epidermis contains about 10 to 20% of moisture and exists in the outermost layer of the human body, thereby suppressing moisture evaporation to the outside of the body and blocking excessive penetration of substances from the outside. The surface of the stratum corneum is covered with a thin natural protective barrier made of sebum from sebaceous glands and sweat from sweat glands to prevent the evaporation of moisture.

However, the moisture in the stratum corneum decreases to make the skin dry and the surface rough, due to various causes, such as artificial temperature control of cooling/heating due to changes in the environment or lifestyle patterns, skin stress due to various stresses and environmental pollution that occur in social life, frequent face washing due to makeup habits, and natural skin aging due to increasing age, etc.

The causes of skin aging may be broadly classified into two types of internal factors such as genetic mutation of cells and tissue changes, and external factors such as ultraviolet rays and humidity. In particular, the aging caused by ultraviolet rays is called photoaging. Reactive oxygen radicals are generated inside cells by ultraviolet rays, and the reactive oxygen promotes the synthesis of proteases (MMP-1, MMP-3, MMP-9, etc.) that degrade collagen, elastin, and the like, which are elastic fibers of the dermal layer, through a signaling system that causes an inflammatory reaction and reduces the elasticity of the dermal layer to cause causes skin wrinkles.

In particular, elastase, which exists in neutrophil granulocytes of the human body, is an enzyme that degrades elastin, a matrix protein important in maintaining skin elasticity in the dermis, and also has collagen-degrading activity. When the elastase is expressed and activated appropriately, the elastase is involved in wound healing and the like, but when the elastase is overexpressed or activated, the elastase degrades elastin in the skin to lose the elasticity of the skin.

In addition, hyaluronic acid (HA), a major component of the extracellular matrix (ECM), is known to be involved not only in moisture retention, maintenance of intercellular spacing, and storage and diffusion of cell growth factors and nutrients, but also in the division, differentiation, and migration of cells. It is known that HA in the skin is mainly synthesized by hyaluronic acid synthase (HAS) on the inner surface of the cell membrane of epidermal keratinocytes and dermal fibroblasts and then accumulated in the ECM. Accordingly, a decrease in HA and defects in the moisture barrier due to decreased expression of HAS may cause skin aging, such as epidermal atrophy, wrinkle formation, decreased skin moisture content, and decreased elasticity.

Retinoic acid, which has been currently used as a skin aging preventive and therapeutic agent, has a weak skin absorption capacity due to fat-soluble properties, so that it is difficult to expect a great effect in promoting collagen synthesis and preventing elastin degradation, it is irritating, and stability problems are emerging due to rapid enzymatic degradation in vivo.

Accordingly, there are still no skin moisturizers or skin wrinkle improving agents that have been recognized as having significant effects, and thus, various studies have been conducted on biologically active substances derived from natural products that are not toxic to the skin while having the effects of maintaining and strengthening skin moisture and improving skin wrinkles.

Meanwhile, agar is the main component of the cell wall of red seaweed and consists of agarose and agaropectin. Agarose consists of a linear chain of alternating 3-O-linked β-D-galactopyranose and 4-O-linked 3,6-anhydro_α-L-galactose unit. Agar has been widely used as a gelling substance in the pharmaceutical, cosmetic, and food industries, and has been used in microorganism culturing media, electrophoresis gel, and chromatography resin. Recently, studies have been reported that agar-derived oligosaccharides exhibited various physiological activities and then, studies have been conducted to be used as potential functional materials in various fields, in addition to using directly existing agar. Agarases may hydrolyze agar and agarose. Based on the mechanism of actions of the agarases, the agarases are divided into α-agarase and β-agarase. The α-agarase hydrolyzes α-1,3-linkages of agarose to generate agarooligosacchride (AOS) and the β-agarase hydrolyzes β-1,4-linkage to generate neoagaro-oligosaccharides (NAOS). The functionality of neoagaro-oligosaccharides, a product of β-agarase of agar, has been reported to include anticancer function, macrophage activation, antioxidant activity, and skin whitening effects.

In the cosmetic industry, demand for products capable of suppressing skin aging is increasing. Skin aging is associated with loss of skin moisture, and hyaluronic acid is a well-known key molecule involved in skin aging. The hyaluronic acid is a polymeric polysaccharide first discovered in the vitreous body of bovine eyes and has a straight structure in which disaccharide units having glycosidic linkage of D-glucuronic acid and N-acetyl-D-glucosamine repeated β-1,4 bonds. The hyaluronic acid may be depolymerized by hyaluronidase and may be useful in preventing skin aging, so that it is very important to maintain homeostasis of hyaluronic acid.

The functionality of oligosaccharides as a hydrolysis product is highly related to properties such as degree of depolymerization (DP) and molecular size. Kobayashi et al. reported the high hygroscopic ability of neoagarobiose (NA2), and thereafter, Ohta et al. reported that neoagarohexaose (NA6) had a higher moisturizing effect than small-molecular oligosaccharides due to higher viscosity. Agar is known to have a very excellent moisture-containing capacity, approximately 3 times higher than the mass. However, due to the gelling properties of agar, it is not easy to formulate cosmetic materials. In addition, to date, there have been no reports on the hyaluronidase inhibitory effect of agar and agar hydrolysis products.

Methods for hydrolyzing polysaccharides include chemical hydrolysis and enzymatic hydrolysis methods. The chemical hydrolysis method has an advantage of a simple process and low production cost, but has a disadvantage of producing monosaccharides and toxic substances and having low yield. On the other hand, the enzymatic hydrolysis method has an advantage of high efficiency, specific action, and high yield, but has the biggest disadvantage of high enzyme production cost.

Therefore, if the agar may be partially hydrolyzed with only a small amount of enzyme to efficiently generate functional oligosaccharides and hydrolysis products, the agar has high applicability to cosmetic materials and pharmaceutical-related industries by reducing production costs, which is the biggest disadvantage of enzymatic hydrolysis.

### SUMMARY

The present inventors found that while searching for natural products with skin moisturizing or skin wrinkle improvement effects, an agar partial hydrolysis product (PHP) obtained by treating agar with a recombinant agarase rGaa16B derived from Gilvimarinus agarilyticus JEA5 exhibited a significant hyaluronidase (HAase) inhibitory effect, and then completed the present disclosure.

The present disclosure has been made in an effort to provide a method for preparing an agar partial hydrolysis product having hyaluronidase inhibitory activity.

The present disclosure has also been made in an effort to provide an agar partial hydrolysis product having hyaluronidase inhibitory activity prepared by the method.

The present disclosure has also been made in an effort to provide a method for improving skin aging or skin condition of a subject, the method comprising administering to the subject an effective amount of the agar partial hydrolysis product.

The present disclosure has also been made in an effort to provide a cosmetic composition for moisturizing skin or improving skin wrinkles including an agar partial hydrolysis product as an active ingredient.

The present disclosure has also been made in an effort to provide a food composition for moisturizing skin or improving skin wrinkles including an agar partial hydrolysis product as an active ingredient.

The present disclosure has also been made in an effort to provide a pharmaceutical composition for preventing or treating wrinkles including an agar partial hydrolysis product as an active ingredient.

The terms used herein are used for the purpose of description only, and should not be construed to be limited. The singular expression includes the plural expression unless the context clearly dictates otherwise. In the present disclosure, it should be understood that term “comprising” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which examples pertain. Terms which are defined in a generally used dictionary should be interpreted to have the same meaning as the meaning in the context of the related art, and are not interpreted as ideal or excessively formal meanings unless otherwise explicitly defined in the present disclosure.

Hereinafter, the present disclosure will be described in more detail.

An exemplary embodiment of the present disclosure provides a method for preparing an agar partial hydrolysis product having hyaluronidase inhibitory activity including partially hydrolyzing agar by adding an agarase and an agar partial hydrolysis product having hyaluronidase inhibitory activity prepared by the method.

In the present disclosure, the partial hydrolysis refers to incomplete hydrolysis until the agar is completely hydrolyzed and homogenized into low molecular substances, and may be achieved by controlling hydrolysis conditions, such as temperature and time. For example, the partial hydrolysis is reacting at 1 to 100° ° C. for 1 to 120 minutes, but is not limited thereto, as long as the agar partial hydrolysis product of the present disclosure may be obtained. In addition, the agar partial hydrolysis product may be used by heating at 80 to 100° C. for 1 to 100 minutes to inactivate the enzyme after the hydrolysis reaction. In addition, the supernatant may be separated and used by performing centrifugation and filtration under reduced pressure.

In addition, for ease of processing, storage, and the like when preparing the agar partial hydrolysis product of the present disclosure, a filtration process, a concentration and purification process, a drying process, a freezing process, a powdering process, etc. may be optionally added.

In an example of the present disclosure, the reaction is terminated after reacting at 55° C. for 5 to 60 minutes and then inactivating the enzyme at 90° ° C. for 10 minutes.

According to the method of the present disclosure, the partial hydrolysis product of the present disclosure, the obtained product refers to a product which is not completely hydrolyzed and homogenized into low molecular materials, but includes high molecular materials of various molecular weights with a polydispersity (Mw/Mn) value of 3 to 8, preferably 5 to 7, and thus, has superior hyaluronidase inhibitory activity to a complete hydrolysis product.

In addition, the agar partial hydrolysis product of the present disclosure generates and includes more neoagarotetraos (NA4) and neoagarohexaose (NA6) than neoagarobiose (NA2).

In the present disclosure, the agar partial hydrolysis product of the present disclosure may be obtained by hydrolyzing the agar with known agarases, more preferably obtained by hydrolyzing the agar with recombinant Gaa16B (rGaa16B) which is an agarase consisting of an amino acid sequence represented by SEQ ID NO: 2.

The agar partial hydrolysis product of the present disclosure obtained through the hydrolysis process as described above is characterized by inhibiting the activity of hyaluronidase. The hyaluronidase degrades HA by hydrolyzing the glycosidic bonds of hyaluronic acid (HA), an important factor in skin moisturization. Accordingly, the agar partial hydrolysis product of the present disclosure achieves the effect of maintaining or enhancing skin moisturization by inhibiting the hyaluronidase activity.

In addition, the hyaluronidase is an enzyme that hydrolyzes hyaluronic acid, and the hyaluronic acid is a substance that plays an important role in the moisturizing function of the skin and has the ability of containing a large amount of moisture, and as a result, when the hyaluronic acid is reduced in the skin, the skin may be dried out to cause wrinkles. Accordingly, the agar partial hydrolysis product of the present disclosure has excellent hyaluronidase inhibitory activity to achieve the effect of improving skin wrinkles or suppressing wrinkle formation.

In other words, the agar partial hydrolysis product of the present disclosure has excellent efficacy in moisturizing the skin, preventing loss of skin elasticity, improving skin wrinkles, and preventing wrinkles.

Accordingly, the agar partial hydrolysis product may be used for various applications, such as cosmetic compositions, food compositions, external skin preparations, and pharmaceutical compositions due to the skin condition improving activity.

In addition, another exemplary embodiment of the present disclosure provides a method for improving skin aging or skin condition of a subject, the method comprising administering to the subject an effective amount of the agar partial hydrolysis product.

The agar partial hydrolysis product of the present disclosure improves the reduction and/or prevention of skin fine lines or skin wrinkles; or the improvement of moisturizing.

The agar partial hydrolysis product is administered as a cosmetic or health functional food or dermopharmaceutical composition.

In addition, another exemplary embodiment of the present disclosure provides a cosmetic composition for moisturizing skin or improving skin wrinkles including an agar partial hydrolysis product as an active ingredient.

The ingredients included in the cosmetic composition of the present disclosure may include ingredients which are generally used in the cosmetic composition in addition to the agar partial hydrolysis product as active ingredients, and for example, include general adjuvants and carriers, such as stabilizers, solublizers, vitamins, pigments, and fragrances.

The cosmetic composition according to the present disclosure may be formulated into external skin preparations or cosmetics by conventional methods. The external skin preparations may include ointments, plasters, sprays, suspensions, emulsions, creams, gels, etc., but are not limited thereto.

The cosmetic composition of the present disclosure may be prepared in any formulation commonly prepared in the art, and examples thereof may include emulsions, creams, lotions, packs, foundations, lotions, essences, hair cosmetics, etc.

Specifically, the cosmetic composition of the present disclosure includes formulations of skin lotion, skin softener, skin toner, astringent, lotion, milk lotion, moisture lotion, nutritional lotion, massage cream, nutritional cream, moisture cream, hand cream, foundation, essence, nutritional essence, pack, soap, cleansing foam, cleansing lotion, cleansing cream, body lotion, and body cleanser.

When the formulation of the present disclosure is the paste, cream, or gel, as a carrier ingredient, animal oils, vegetable oils, wax, paraffin, starch, tragacanth, cellulose derivatives, polyethylene glycol, silicone, bentonite, silica, talc, zinc oxide or the like may be used.

When the formulation of the present disclosure is the powder or spray, as the carrier ingredient, lactose, talc, silica, aluminum hydroxide, calcium silicate or polyamide powder may be used, and particularly, in the case of the spray, additionally, a propellant such as chlorofluorohydrocarbon, propane/butane or dimethyl ether may be included.

When the formulation of the present disclosure is the solution or emulsion, as the carrier ingredient, a solvent, a solubilizing agent or an emulsifying agent may be used. For example, the carrier ingredient includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan.

When the formulation of the present disclosure is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

In addition to the active ingredients, the cosmetic composition of the present disclosure may further include one or more ingredients that help in improving skin condition, including ingredients for maintaining and enhancing skin moisturization or improving wrinkles, which have the same or similar functions. The ingredients include hyaluronic acid, butylene glycol, glycerin, amino acid, trehalose, kojic acid and derivatives thereof, arbutin, ascorbic acid and derivatives thereof, hydroquinone and derivatives thereof, resorcinol, 2,7-dinitroindazole, adenosine, retinol, retinyl palmitate, polyethoxylated retinamide, yeast, dipeptide, palmitoyl oligopeptide & palmitoyl tripeptide-7, acetyl hexapeptide, epidermal growth factor (EGF), or plant extracts such as tangerine, rice, licorice, shea butter, aloe vera, coconut, olive, and avocado, but are not limited thereto.

In addition, the present disclosure may provide a pack including the cosmetic composition for moisturizing skin or improving skin wrinkles including the agar partial hydrolysis product as an active ingredient.

The pack is preferably prepared in any one formulation selected from a mask pack, a sleeping pack, a cleansing pack, a wash off pack, and a peel off pack, but is not limited thereto.

In addition, yet another exemplary embodiment of the present disclosure provides a food composition or health functional food for moisturizing skin or improving skin wrinkles including an agar partial hydrolysis product as an active ingredient.

As used in the present disclosure, the “food” means natural products or processed products containing one or more nutrients, and preferably, means a condition that may be eaten directly through some processing processes, and as a general meaning, may include all of foods, food additives, functional foods and beverages.

The food composition according to the present disclosure includes all forms, such as functional foods, nutritional supplements, health foods, and food additives. The type of food composition may be prepared in various forms according to general methods known in the art.

For example, as the health food, the agar partial hydrolysis product itself of the present disclosure may be prepared in the form of tea, juice, and drinks, to be drunk, or taken in by granulation, encapsulation and powder. In addition, the agar partial hydrolysis product of the present disclosure may be prepared in the form of a composition by mixing known substances or active ingredients known to have an allergy improvement or treatment effect.

Further, the functional food may be prepared by adding the agar partial hydrolysis product of the present disclosure to beverages (including alcoholic beverages), fruits and processed foods thereof (e.g., canned fruit, bottled food, jam, marmalade, etc.), fish, meat and processed foods thereof (e.g., ham, sausage, corned beef, etc.), bread and noodles (e.g., udon, buckwheat noodles, ramen, spaghetti, macaroni, etc.), fruit juice, various drinks, cookies, sweets, dairy products (e.g., butter, cheese, etc.), edible vegetable oil, margarine, vegetable protein, retort foods, frozen foods, various seasonings (e.g., soybean paste, soy sauce, sauce, etc.), etc.

The preferred content of the agar partial hydrolysis product of the present disclosure in the food composition of the present disclosure is not limited thereto, but may be, for example, 0.01 to 80 wt % of finally prepared food, preferably 0.01 to 50 wt % of the finally prepared food.

In addition, in order to use the agar partial hydrolysis product of the present disclosure in the form of food additives, the agar partial hydrolysis product may be prepared and used in the form of powder or concentrate.

In addition, yet another exemplary embodiment of the present disclosure provides a pharmaceutical composition for preventing or treating wrinkles including an agar partial hydrolysis product as an active ingredient.

The agar partial hydrolysis product of the present disclosure has excellent skin moisturizing and wrinkle inhibiting effects, and thus, may be usefully used as an active ingredient in the pharmaceutical composition for preventing or treating wrinkles.

When the composition of the present disclosure is the pharmaceutical composition for preventing or treating wrinkles, the pharmaceutical composition of the present disclosure may further include a pharmaceutically acceptable additive. At this time, the pharmaceutically acceptable additive may be used with starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar and the like. The pharmaceutically acceptable additive according to the present disclosure is preferably included in an amount of 0.1 to 90 parts by weight based on the composition, but is not limited thereto.

The pharmaceutical composition of the present disclosure may be administered in various oral or parenteral formulations during actual clinical administration, but may be prepared using commonly used diluents or excipients, such as fillers, extenders, binders, wetting agents, disintegrants, surfactants, etc., for formulations. Suitable formulations known in the art are preferably used with formulations disclosed in the literature (Remington's Pharmaceutical Science, last, Mack Publishing Company, Easton PA).

Solid formulations for the oral administration include tablets, pills, powders, granules, capsules, and the like, and the solid formulations may be prepared by mixing at least one excipient, for example, starch, calcium carbonate, sucrose or lactose, gelatin, and the like. Further, lubricants such as magnesium stearate and talc may be used in addition to simple excipients. In addition, liquid formulations for the oral administration may correspond to suspensions, oral liquids, emulsions, syrups, and the like, and may include various excipients, for example, wetting agents, sweeteners, aromatic agents, a preservatives, and the like, in addition to water and liquid paraffin which are commonly used as simple diluents.

The formulations for the parenteral administration include a sterile aqueous solution, a non-aqueous solvent, a suspension, an emulsion, a lyophilizing agent, and a suppository. As the non-aqueous solvent and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

In addition, the dose of the pharmaceutical composition of the present disclosure may vary depending on a formulation method, an administration method, an administration time, and/or an administration route of the pharmaceutical composition, and vary depending on various factors including the type and degree of a response to be achieved by administration of the pharmaceutical composition, the type, age, body weight, and general health condition of a subject to be administered, symptoms or severity of a disease, sex, diet, excretion, drugs used simultaneously or separately for the corresponding subject, other composition ingredients, and the like, and similar factors well-known in the field of medicine. In addition, an effective dose for a desired treatment may be easily determined and prescribed by those skilled in the art.

The dose of the pharmaceutical composition of the present disclosure may be administered at a concentration of, for example, preferably 0.01 to 5 mg/kg, more preferably 0.05 to 0.4 mg/kg, and much more preferably 0.1 to 0.35 mg/kg, but the dose does not limit the scope of the present disclosure in any aspect.

The administration route and the administration method of the pharmaceutical composition of the present disclosure may be independent of each other, and the method is not particularly limited, and may be any administration route and administration method so long as the pharmaceutical composition may reach a corresponding site.

The pharmaceutical composition may be administered orally or parenterally. The parenteral administration method includes, for example, intravenous administration, intraperitoneal administration, intramuscular administration, transdermal administration, subcutaneous administration, or the like.

The pharmaceutical composition of the present disclosure may be used alone or in combination with surgery, radiation therapy, hormone therapy, chemotherapy, and methods of using biological response modifiers, for prevention or treatment of wrinkles.

According to the exemplary embodiments of the present disclosure, the agar partial hydrolysis product excellently inhibits the activity of hyaluronidase, which degrades hyaluronic acid (HA), an important factor in skin moisturization, and thus may be variously used in cosmetics, food, or medicine to maintain and enhance skin moisturization, maintain skin elasticity, improve skin wrinkles, as well as promote skin regeneration and prevent aging.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows SDS-PAGE results of recombinant agarose and rGaa16B protein.

M: Molecular mass marker (Thermo scientific, USA). Lane 1: whole cell lysates from E. coli BL21 (DE3) before induction; lane 2: whole cell lysates after IPTG induction; lane 3: total soluble cellular extract after induction; lane 4: total insoluble cellular extract after induction; lane 5: purified rGaa16Bc.

FIG. 2A-B shows thin layer chromatography results of PHP sample (FIG. 2A) and CHP (FIG. 2B). D-galactose (G), neoagarobiose (NA2), neoagarotetraos (NA4) and neoagarohexaose (NA6) were used as standards.

### DETAILED DESCRIPTION

Hereinafter, the present disclosure will be described in detail by Examples. However, the following Examples are just illustrative of the present disclosure, and the contents of the present disclosure are not limited to the following Examples.

### Example 1. Expression and Purification of Recombinant Agarase

The present inventors used Gaa16B (NCBI accession No. KP716980) from Gilvimarinus agarilyticus JEA5 to produce Gaa16B previously known as a recombinant agarase necessary for preparing the agar hydrolysis product of the present disclosure.

This was briefly described as follows:

AccuPrep® Genomic DNA Extraction Kit (Bioneer, Korea Rep) genomic DNA was separated from G. agarilyticus JEA5 (KCCM43129).

To amplify only a catalytic domain part excluding signal peptide and carbohydrate binding domain (CBM) sites, Gaa16B-F (5′-TTC AGA ATT CGG ATC GCC GAC TGG GAC GGC TTA-3′) and Gaa16B-R (5′-TTG CCT GCA GGT CGA CTA GGT AAT GTC GTT ATC GCC ATT GT-3′) primers including both end sequences of an amplification site of Gaa16B and a 15 bp sequence of a cloning site of a pMal-c2x vector were prepared, and a catalytic domain region of Gaa16B was amplified by polymerase chain reaction (PCR) using the primers. The PCR reaction was performed by using 1 μL of genomic DNA (200 ng/μL) as a template, and adding 35.5 μL of distilled water in 5 μL 10X Ex Taq buffer, 1 μL forward and reverse primers (20 pmol each), 4 μL 2.5 mM dNTPs, and Ex Taq DNA polymerase (3 U) with to become a total of 50 μL. PCR conditions were initial denaturation at 94° C. for 5 minutes, 30 cycles of reaction of 94° C. for 30 seconds, 58° C. for 30 seconds, and 72° ° C. for 1 minute and 30 seconds, and final extension at 72ºC for 5 minutes. The PCR reaction was performed using TaKaRa PCR Thermal cycler Dice Gradient (Takara Bio Inc., Japan). PCR amplification products were purified using AccuPrep® Gel Purification Kit (Bioneer, Korea Rep) and then cloned into a pMal-c2x expression vector digested with restriction enzymes BamHI and SalI using an Ez-Fusion™ Cloning Kit (Enzynomics, Korea Rep). The recombinant plasmid was transformed into E. coli DH5α cells, and purified using an AccuPrep® Nano-Plus Plasmid Mini Extraction Kit (Bioneer, Korea Rep) and then transformed again into E. coli BL21 (DE3).

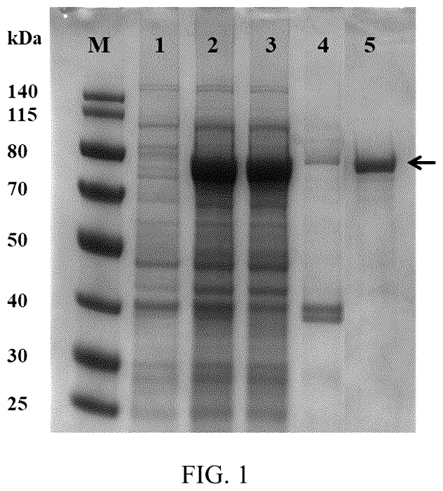

E. coli cells cloned with Gaa16B were inoculated into 5 mL of Luria broth (LB) added with 100 mg/mL of ampicillin and cultured overnight at 37° C., re-inoculated in 250 mL of a new medium and cultured to be a mid-logarithmic phase (OD600 nm=0.6 to 0.7), and then added with 0.05 mM of isopropyl-β-D-thiogalactopyranoside (IPTG) to induce expression of the recombinant protein at 20° C. for 20 hours. The E. coli cells in which recombinant protein expression was induced were centrifuged at 8000×g for 15 minutes, the supernatant was removed, and then the cells were resuspended in a column buffer (200 mM NaCl and 20 mM Tris-HCl), and left at −20° C. overnight. Frozen cells were thawed on ice, lysed by sonication, and centrifuged at 4° C. and 11,000 rpm for 15 minutes to collect the supernatant. The recombinant protein was purified from the supernatant using the pMal™ Protein Fusion and Purification System (New England Biolab, UK), and the expression of the recombinant protein was identified using SDS-PAGE (FIG. 1). The concentration of the purified protein was measured using a BCA protein assay reagent kit (Thermo Fisher Scientific Inc., Waltham, MA, USA).

An nucleotide sequence of the obtained recombinant Gaa16B (rGaa16B), which was an agarase with agar-degrading ability, was represented by SEQ ID NO: 1, and an amino acid sequence thereof was represented by SEQ ID NO: 2.

### Example 2. Preparation of Agar Partial Hydrolysis Product (PHP)

The present inventors prepared an agar partial hydrolysis product (PHP) using rGaa16B of the present disclosure obtained in Example 1 in edible agar (trace agar, Korea Rep).

This was briefly described as follows:

After the agar was dissolved in distilled water at a concentration of 1%, rGaa16B was treated at a concentration of 0.1 unit/mL and reacted at 55° C. for 5, 10, 20, 30, and 60 minutes, respectively, and then inactivated at 90° ° C. for 10 minutes to complete the reaction. Samples were referred to as PHP5, PHP10, PHP20, PHP30, and PHP60 according to a reaction time, respectively. The sample in which the agar was completely hydrolyzed by reacting at 55° C. overnight was referred to as CHP.

To identify PHP degraded by rGaa16B, the PHP was identified through thin layer chromatography (TLC). D-galactose (Sig-ma-Aldrich, USA), neoagarobiose (NA2), neoagarotetraose (NA4), and neoagarohexaose (NA6) (Carbosynth, UK) were used as standards. PHP and CHP were developed on a silica gel 60 TLC plate. The developing solvent was butanol:acetic acid:dH₂O (2:1:1 [v/v]). An orcinol dip reagent (dissolving 80 mg of orcine monohydrate in 160 mL of acetone and then adding 8 mL of sulfuric acid) was applied and visualized by heating in a 110° C. dry oven for 10 minutes.

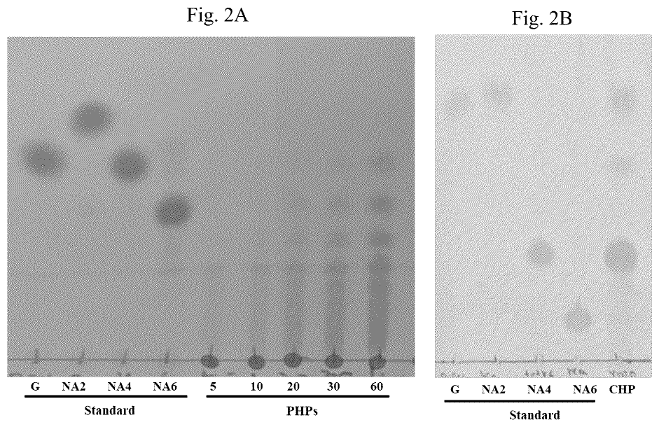

As a result, as shown in FIG. 2A, the amount of low-molecular polysaccharides gradually increased in PHP5, 10, 20, 30, and 60 as the reaction time increased. In PHP5, spots corresponding to NA4 and NA6 were barely shown, but as the reaction time increased, the intensity of each spot became stronger. In PHP60, which was hydrolyzed for 60 minutes, NA4 and NA6 were clearly shown, and various spots above NA6 were clearly observed. In addition, as shown in FIG. 2B, in the case of completely hydrolyzed CHP, it was confirmed that most of CHP was degraded into NA4, and NA2 was faintly observed.

### Example 3. Characteristics of Agar Partial Hydrolysis Product (PHP)

The present inventors confirmed the characteristics of the agar partial hydrolysis product (PHP) of the present disclosure prepared in Example 2.

This was briefly described as follows:

Gel permeation chromatography (GPC) was performed to measure the average molecular weight (Mw) and polydispersity (Mw/Mn) of the partial hydrolysis product PHPs. In the Waters e2695 instrument, a column was used with an Ultrahydrogel 120, 250, 500, 1000 (Waters, USA) column (7.8 mm×300 mm) and a refractive index detector (RID) was used. 5 mg of freeze-dried PHP and CHP powders were dissolved in 1 mL of distilled water, filtered using a 0.45 μm filter, and 100 μL of PHP and CHP were injected at 35° C. The mobile phase was 0.1 M NaNO₃ flowed at a flow rate of 1 mL/min, and 0.3 mg/mL of pullulan was used as the standard.

A calibration curve of standard was plotted as the logarithm of relative molecular weight (Mw, Da) versus the retention time (t, min), the regression equation was obtained, and the Mw of PAPs and CHP were determined based on the retention time of polysaccharide solution. All data were processed using Empower 3 Chromatography Data Software (Waters, USA).

The GPC results of PHP and CHP were shown in Table 1. The average molecular weights (Mw) of the main peaks of PHP5, 10, 20, 30, and 60 were variously expressed as 22,686, 16,636, 14,948, 14,109, and 10,635 Da, respectively. On the other hand, the average molecular weight of completely hydrolyzed CHP was 459 Da.

In addition, the polydispersity was calculated as Mw/Mn, and if all molecules had the same molecular weight, Mw=Mn, and as a result, Mw/Mn closed to 1. Based on this, the polydispersity of the PHP sample was 5.5 to 6.4, which proved that hydrolysis products of various sizes were distributed, while the polydispersity of CHP was 1.034, which may be seen that the CHP was uniform due to its high purity.

In addition, in order to identify the viscosity of PHP and CHP, the average value was obtained by using Viscometer DV2TLVTJO (AMETEK BROOKFIELD, USA) and measuring three times at a rotation speed of 50 rpm at room temperature using Spindle No. 31.

As a result, it was confirmed that PHP had higher viscosity than CHP.

TABLE 1

| Sample | MP | Mw | | | Viscosity |
|---|---|---|---|---|---|
| name | (Daltons) | (Daltons) | Polydispersity | % area | (cp) |
| PHP5 | 22,686 | 102,206 | 5.520 | 70.01 | 187.7 ± 2.1 |
| PHP10 | 16,636 | 115,319 | 6.141 | 71.13 | 96.0 ± 5.7 |
| PHP20 | 14,948 | 109,822 | 5.843 | 69.12 | 95.4 ± 4.8 |
| PHP30 | 14,109 | 92,705 | 5.761 | 66.81 | 89.9 ± 2.2 |
| PHP60 | 10,635 | 88,617 | 6.494 | 59.85 | 19.9 ± 0.7 |
| CHP | 459 | 473 | 1.034 | 84.28 | 1.4 ± 0.3 |

### Example 4. Hyaluronidase (HAase) Inhibitory Activity of Agar Partial Hydrolysis Product (PHP)

The present inventors identified hyaluronidase (HAase) inhibitory activity of the agar partial hydrolysis product (PHP) of the present disclosure.

This was briefly described as follows:

The hyaluronidase (HAase) inhibitory activity was identified by measuring N-acetylglucosamine produced by degradation of hyaluronic acid by hyaluronidase. 12.5 μL of HAase (8 mg/mL) dissolved in 0.1 M acetate buffer (pH 3.5) was mixed with 12.5 μL of agar partial hydrolysis product (PHP) at a concentration of 9 mg/mL and reacted at 37° C. for 20 minutes. To activate the HAase, the mixture was added with 25 μL of 12.5 mM CaCl₂) and reacted at 37ºC for 20 minutes, and added with 62.5 μL of hyaluronic acid (2.4 mg/ml) dissolved in 0.1 M acetate buffer (pH 3.5) and reacted at 37° ° C. for 40 minutes again. To stop the substrate-enzyme reaction, the reaction solution was added with 25 μl of 0.4 N NaOH and 25 μl of 0.4 N potassium tetraborate, reacted at 100° C. for 3 minutes, and then cooled. The reaction solution was added with 750 μl of a DMAB solution and cultured at 37ºC for 20 minutes, and the absorbance was measured at 540 nm. The inhibitory activity was calculated using the following equation.

$$Hyaluronidase Inhibition ( \% ) = \frac{ODc - ODs}{ODc} \times 100$$

As a result, as shown in Table 2, when the hyaluronidase inhibition rates (%) and half maximal effective concentrations (IC₅₀) of PHP and CHP were confirmed, IC₅₀ of PHP5, 10, 20, 30, and 60 were 0.47, 0.40, 0.31, 0.41, and 0.57, respectively. At the beginning of hydrolysis, the IC₅₀ value decreased as the hydrolysis time increased, and the lowest IC₅₀ was shown at PHP20, and thereafter, as the reaction time increased, the IC₅₀ value tended to increase. As a result, it was confirmed that PHP20 most effectively inhibited hyaluronidase. The IC₅₀ for PHP5 to 60 was 0.47 to 0.57 mg/mL, and when treated at a concentration of 1 mg/mL, all samples showed an inhibitory effect of 60% or more. On the other hand, in the case of CHP, the IC₅₀ value was not measured and only the inhibitory effect of 7.46% was shown at a concentration of 1 mg/mL.

In other words, when the agar was completely hydrolyzed, there was almost no hyaluronidase inhibitory activity, but when the agar was partially hydrolyzed, the hyaluronidase inhibitory activity was significantly increased.

TABLE 2

| | Sample solution | Inhibition | IC₅₀ |
|---|---|---|---|
| Sample Name | (mg/mL) | (%) | (mg/mL) |
| PHP5 | 1 | 63.94 ± 0.27 | 0.47 ± 0.04 |
| PHP10 | 1 | 64.02 ± 0.36 | 0.40 ± 0.02 |
| PHP20 | 1 | 63.65 ± 0.28 | 0.31 ± 0.05 |
| PHP30 | 1 | 62.81 ± 0.50 | 0.41 ± 0.03 |
| PHP60 | 1 | 63.29 ± 0.39 | 0.57 ± 0.10 |
| CHP | 1 | 7.46 ± 0.12 | ND* |
| DSCG | 1 | 65.32 ± 0.20 | 0.028 ± 0.01 |

DSCG: Disodium cromoglycate, Anti-allergy and anti-inflammatory drug known as hyaluronidase inhibitor

In conclusion, the agar partial hydrolysis product (PHP) obtained by Gaa16B treatment of G. agarilyticus JEA5 of the present disclosure exhibited an excellent inhibitory effect on hyaluronidase, which degraded HA by hydrolyzing the glycosidic bonds of hyaluronic acid (HA), an important factor in skin moisturization. Therefore, it is suggested that the agar partial hydrolysis product (PHP) of the present disclosure may exhibit an effect of improving overall skin condition, and based on this, the agar partial hydrolysis product (PHP) may be applied to various technical fields, such as functional cosmetic compositions, functional food compositions, and pharmaceutical compositions for preventing and treating wrinkles.

As described above, specific parts of the present disclosure have been described in detail, and it will be apparent to those skilled in the art that these specific techniques are merely preferred embodiments, and the scope of the present disclosure is not limited thereto. Therefore, the substantial scope of the present disclosure will be defined by the appended claims and their equivalents.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

[Sequence List Text]

Electronic file attachment

What is claimed is:

1. A method for preparing an agar partial hydrolysis product having hyaluronidase inhibitory activity, the method comprising partially hydrolyzing agar by adding an agarase.

2. The method for preparing the agar partial hydrolysis product of claim 1, wherein the agarase consists of an amino acid sequence represented by SEQ ID NO: 2.

3. The method for preparing the agar partial hydrolysis product of claim 1, wherein the partial hydrolysis is reacting at 1 to 100° C. for 1 to 120 minutes.

4. The method for preparing the agar partial hydrolysis product of claim 3, wherein after the reaction, the agarase is inactivated at 80 to 100° C. for 1 to 100 minutes to complete the reaction.

5. An agar partial hydrolysis product having hyaluronidase inhibitory activity prepared by the method of claim 1.

6. The agar partial hydrolysis product of claim 5, wherein the agar partial hydrolysis product comprises more neoagarotetraos (NA4) and neoagarohexaose (NA6) than neoagarobiose (NA2).

7. The agar partial hydrolysis product of claim 5, wherein the agar partial hydrolysis product has a polydispersity (Mw/Mn) of 3 to 8.

8. A method for improving skin aging or skin condition of a subject, the method comprising administering to the subject an effective amount of the agar partial hydrolysis product of claim 5.

9. The method of claim 8, wherein the improvement is the reduction and/or prevention of fine lines or wrinkles.

10. The method of claim 8, wherein the improvement is the improvement of moisturizing.

11. The method of claim 8, wherein the agar partial hydrolysis product is administered as a cosmetic or health functional food or pharmaceutical composition.
